(19) United States
(12) Patent Application Publication
(10) Pub. No.: US 2023/0130552 A1
(43) Pub. Date: Apr. 27, 2023

(54) ENDOSCOPE SYSTEM AND ENDOSCOPE

(71) Applicant: OLYMPUS CORPORATION, Tokyo (JP)

(72) Inventors: Chikayoshi MEGURO, Hachioji-shi (JP); Tatsuhiko SUZUKI, Koganei-shi (JP); Yui KAWANISHI, Koganei-shi (JP); Yukiko ASAI, Hachioji-shi (JP); Mariko BAMBA, Musashino-shi (JP)

(73) Assignee: OLYMPUS CORPORATION, Tokyo (JP)

(21) Appl. No.: 17/969,865

(22) Filed: Oct. 20, 2022

Related U.S. Application Data
(60) Provisional application No. 63/272,338, filed on Oct. 27, 2021.

(51) Int. Cl. A61B 1/00; A61B 1/12

(52) U.S. Cl. CPC A61B 1/0016; A61B 1/12

(57) ABSTRACT

An endoscope system includes: a drive cable; an endoscope driven by the drive cable that has been connected; a drive device that electrically drives the connected drive cable; a trolley equipped with the drive device; and a stand that supports a connector that detachably connects the drive cable and the endoscope, and is capable of being installed at a position away from the trolley.

### CROSS-REFERENCE TO RELATED APPLICATIONS

The present application claims priority based on U.S. Patent Provisional Application No. 63/272,338 provisionally filed in the United States on Oct. 27, 2021, the contents of which are incorporated herein by reference.

### BACKGROUND

### Technical Field

The present disclosure relates to an endoscope system and an endoscope.

### Background Art

Conventionally, an endoscope has been used for observation and treatment in luminal organs such as the digestive tract. There is a demand for an endoscope system that can perform observations and treatments using an endoscope more efficiently. In the electric curved endoscope described in U.S. Pat. No. 798,790 (Patent Document 1), since the insertion portion is electrically driven to be curved, observation and treatment by the endoscope can be efficiently performed.

It is desirable that an endoscope system equipped with an electrically driven endoscope be easy to handle and that the electrically driven insertion portion, the drive portion for electrically driving the insertion portion, the operation portion to which the operator's operation is input, and the like be easy to reprocess (clean).

### SUMMARY

According to a first aspect of the present invention, an endoscope system includes: a drive cable; an endoscope driven by the drive cable that has been connected; a drive device that electrically drives the connected drive cable; a trolley equipped with the drive device; and a stand that supports a connector that detachably connects the drive cable and the endoscope, and is capable of being installed at a position away from the trolley

### BRIEF DESCRIPTION OF THE DRAWINGS

FIG. 1 is an overall view of an endoscope system according to a first embodiment.

FIG. 2 is a diagram showing an endoscope and an operation device of the endoscope system used by an operator.

FIG. 3 is a diagram showing an insertion portion of the endoscope.

FIG. 4 is a diagram showing a part of a curved portion of the endoscope as a cross-sectional view.

FIG. 5 is an enlarged view of a nodal ring of the curved portion in region E shown in FIG. 4.

FIG. 6 is a cross-sectional view of the curved portion along the C1-C1 line of FIGS. 4 and 5.

FIG. 7 is a perspective view of a connector of the endoscope system before mounting.

FIG. 8 is a functional block diagram of a drive device of the endoscope system.

FIG. 9 is a functional block diagram of a video control device of the endoscope system.

FIG. 10 is a diagram showing a trolley or the like to which a drape is applied.

FIG. 11 is a diagram showing the connector placed near a patient.

FIG. 12 is an overall view of the endoscope system according to a second embodiment.

FIG. 13 is a perspective view of a connector of the endoscope system before mounting.

FIG. 14 is a diagram showing a method of using the endoscope system according to a third embodiment.

FIG. 15 is a diagram showing a way of using the endoscope system.

FIG. 16 is a diagram showing a way of using the endoscope system.

FIG. 17 is a diagram showing a way of using the endoscope system.

FIG. 18 is a diagram showing a way of using the endoscope system.

FIG. 19 is a diagram showing a way of using the endoscope system.

FIG. 20 is a diagram showing a way of using the endoscope system.

FIG. 21 is a diagram showing a way of using the endoscope system.

FIG. 22 is a diagram showing a way of using the endoscope system.

FIG. 23 is a diagram showing a trolley used in a conventional endoscope system.

FIG. 24 is a diagram showing a trolley or the like to which a drape is applied.

FIG. 25 is a diagram showing an endoscope system according to the second embodiment to which a drape is applied.

### DETAILED DESCRIPTION OF THE PREFERRED EMBODIMENTS

### First Embodiment

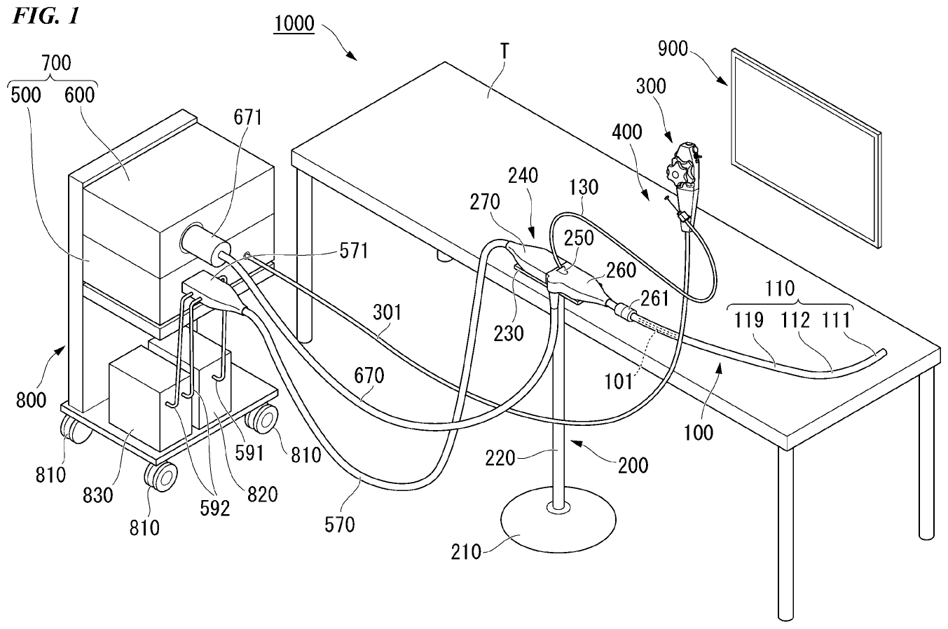

An endoscope system 1000 according to a first embodiment of the present invention will be described with reference to FIGS. 1 to 25. FIG. 1 is an overall view of the endoscope system 1000 according to the present embodiment.

### [Endoscope System 1000]

The endoscope system 1000 is a medical system used to observe and treat the inside of a patient P lying on an operating table T. The endoscope system 1000 includes an endoscope 100, a stand 200, an operation device 300, a treatment tool 400, a drive device 500, a video control device 600, a trolley 800, and a display device 900. A drive cable 570 is connected to the drive device 500. A video control cable 670 is connected to the video control device 600.

The endoscope 100 is a device that is inserted into the lumen of patient P to observe and treat the affected area. The endoscope 100 is connected to the drive device 500 and the video control device 600 via a connector 240 included in the stand 200. An internal path 101 is formed inside the endoscope 100. In the following description, in the endoscope 100, the side inserted into the lumen of the patient P is referred to as “distal end side A1”, and the side connected to the connector 240 is referred to as “proximal end side A2”.

The stand 200 can be arranged at a position away from the trolley 800, and has the connector 240 for connecting the endoscope 100 to the drive cable 570 and the image control cable 670.

The operation device 300 is detachably connected to the drive device 500 via an operation cable 301. The operation device 300 may be able to communicate with the drive device 500 by wireless communication instead of wired communication. A surgeon S can electrically drive the endoscope 100 by operating the operation device 300.

The treatment tool 400 is a device that inserts the internal path 101 of the endoscope 100 into the lumen of the patient P to treat the affected area. In FIG. 1, the treatment tool 400 is inserted into the internal path 101 of the endoscope 100 via an extension channel tube 130 connected to a forceps opening 250 formed in the connector 240.

The drive device 500 is detachably connected to the drive cable 570. The drive device 500 is connected to the endoscope 100 via the drive cable 570 and the connector 240. Further, the drive device 500 is detachably connected to the operation device 300. The drive device 500 drives the endoscope 100 by electrically driving the drive cable 570 using a built-in motor based on the operation input to the operation device 300. Further, the drive device 500 drives a built-in pump or the like based on the operation input to the operation device 300 to cause the endoscope 100 to perform air supply suction.

The video control device 600 is detachably connected to the video control cable 670. The video control device 600 is connected to the endoscope 100 via the image control cable 670 and the connector 240, and acquires a captured image from the endoscope 100. The video control device 600 causes the display device 900 to display the captured image acquired from the endoscope 100, the GUI image for the purpose of providing information to the operator, and the CG image.

The drive device 500 and the video control device 600 constitute a control device 700 that controls the endoscope system 1000. The control device 700 may further include peripheral devices such as a video printer. The drive device 500 and the video control device 600 may be an integrated device.

The trolley 800 is a trolley having wheels 810, and is equipped with the control device 700 (drive device 500 and video control device 600). The trolley 800 may be equipped with, for example, only the drive device 500. Further, the trolley 800 may have a hanger on which the operation device 300 and the like can be suspended.

In the present embodiment, the trolley 800 includes a water supply tank 820 that stores a liquid such as sterilized water used for water supply by the endoscope 100, and a drain tank 830 that collects blood and the like discharged to the outside of the body by suction with the endoscope 100.

The display device 900 is a device capable of displaying an image such as an LCD. The display device 900 is connected to the video control device 600 via a display cable.

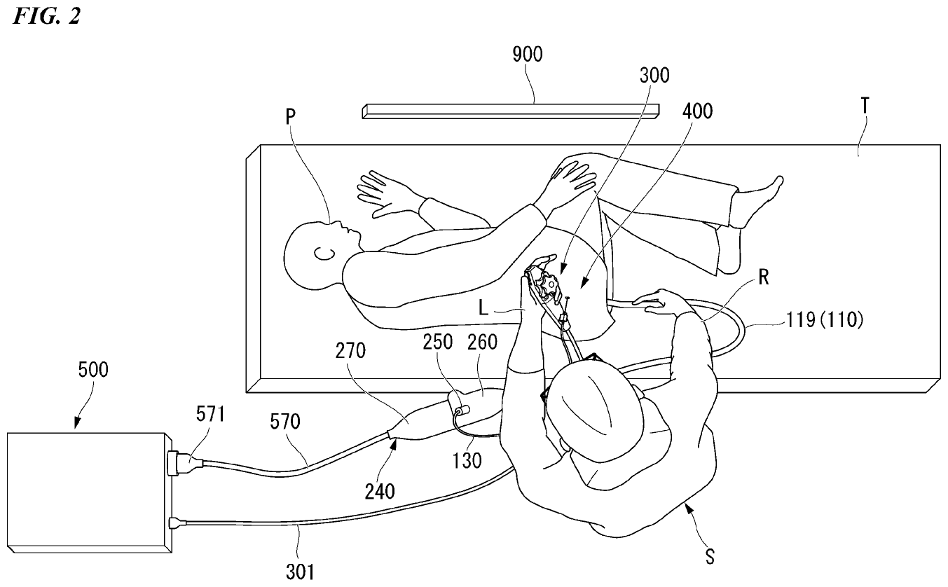

FIG. 2 is a diagram showing the endoscope 100 and the operation device 300 used by the surgeon S.

For example, the surgeon S operates the operation device 300 with the left hand L, while, for example, observing the captured image displayed on the display device 900, while operating the endoscope 100 inserted into the lumen from the anus of patient P with the right hand R. Since the endoscope 100 and the operation device 300 are separated, the operator S can operate the endoscope 100 and the operation device 300 independently without being affected by each other.

Next, each device of the endoscope system 1000 will be described in detail.

### [Endoscope 100]

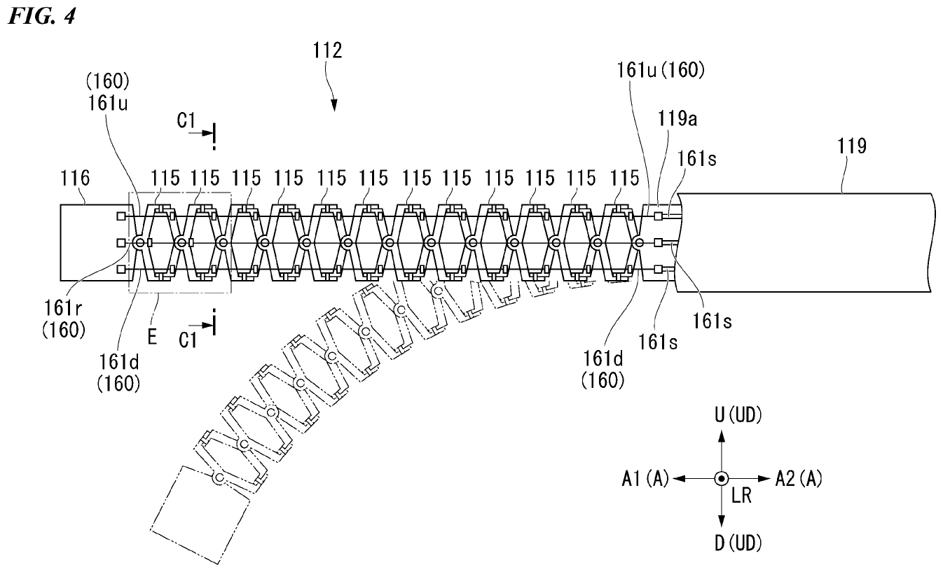

As shown in FIG. 1, the endoscope 100 includes an insertion portion 110, a curved wire 160 (see FIG. 4), and an internal component 170 (see FIG. 4). The proximal end of the endoscope 100 is connected to the connector 240.

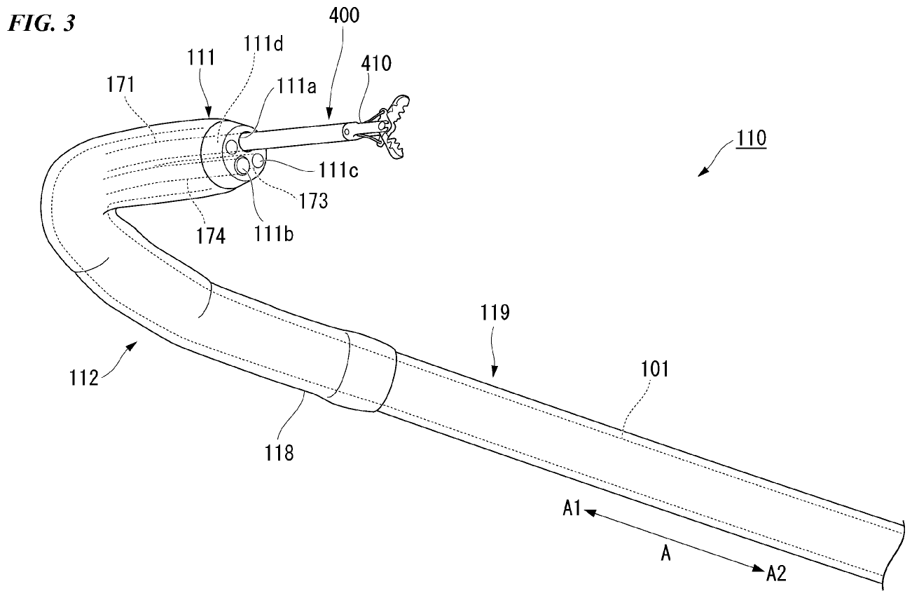

FIG. 3 is a diagram showing the insertion portion 110 of the endoscope 100.

Inside the endoscope 100, the internal path 101 extending from the distal end end of the insertion portion 110 to the proximal end along the longitudinal direction A of the endoscope 100 is formed. The curved wire 160 and the internal structure 170 are inserted into the internal path 101. The internal structure 170 includes a channel tube 171, an imaging cable 173, and a light guide 174.

The insertion portion 110 is an elongated long member that can be inserted into the lumen. The insertion portion 110 has a distal end portion 111, a curved portion 112, and an internal soft portion 119. The distal end portion 111, the curved portion 112, and the internal soft portion 119 are connected in order from the distal end side.

The distal end portion 111 is formed in a substantially cylindrical shape by metal or the like. As shown in FIG. 3, the distal end portion 111 has an opening portion 111a, an illumination portion 111b, and an imaging portion 111c. The opening 111a is an opening that communicates with the channel tube 171. As shown in FIG. 3, a treatment portion 410 such as a gripping forceps provided at the distal end of the treatment tool 400 through which the channel tube 171 is inserted is recessed from the opening 111a.

The illumination portion 111b is connected to the light guide 174 that guides the illumination light, and emits illumination light that illuminates the imaging target. The imaging portion 111c includes an imaging element such as CMOS, and captures an image of an imaging target. The imaging signal is sent to the video control device 600 via the imaging cable 173 and the image control cable 670.

FIG. 4 is a diagram showing a part of the curved portion 112 as a cross-sectional view.

The curved portion 112 has a plurality of knot rings (also referred to as curved pieces) 115, a distal end portion 116 connected to the distal ends of the plurality of knot rings 115, and an outer sheath 118 (see FIG. 3). The distal end portion 116, the plurality of knot rings 115, and a distal end portion 119a of the internal soft portion 119 are connected in the longitudinal direction A inside the outer sheath 118. The shape and number of the knot rings 115 included in the curved portion 112 are not limited to the shape and number of the knot rings 115 shown in FIG. 4.

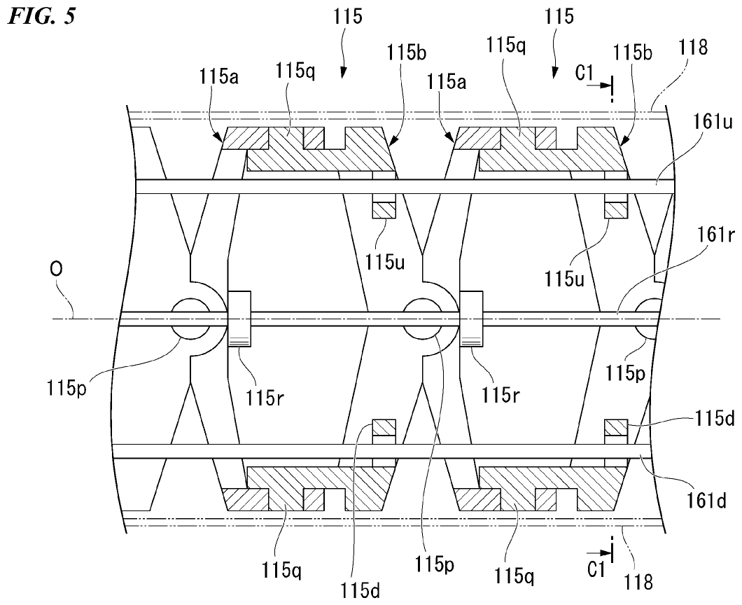

FIG. 5 is an enlarged view of the nodal ring 115 in the region E shown in FIG. 4. The knot ring 115 is a short cylindrical member made of metal. The plurality of knot rings 115 are connected so that the internal space of the adjacent knot rings 115 becomes a continuous space.

Adjacent knot rings 115 are rotatably connected by a first rotation pin 115p in the vertical direction (also referred to as “UD direction”) perpendicular to the longitudinal direction A.

The knot ring 115 has a first node ring 115a on the distal end side and a second node ring 115b on the proximal end side. The first node ring 115a and the second node ring 115b are rotatably connected by a second rotation pin 115q in the left-right direction (also referred to as “LR direction”) perpendicular to the longitudinal direction A and the UD direction.

The first rotation pin 115p of the knot ring (curved piece) 115 is rotatable about a rotation axis extending in the LR direction. The second rotation pin 115q of the nodal ring (curved piece) 115 is rotatable about a rotation axis extending in the UD direction.

The first node ring 115a and the second node ring 115b are alternately connected by the first rotation pin 115p and the second rotation pin 115q, and the curved portion 112 can be bent in a desired direction.

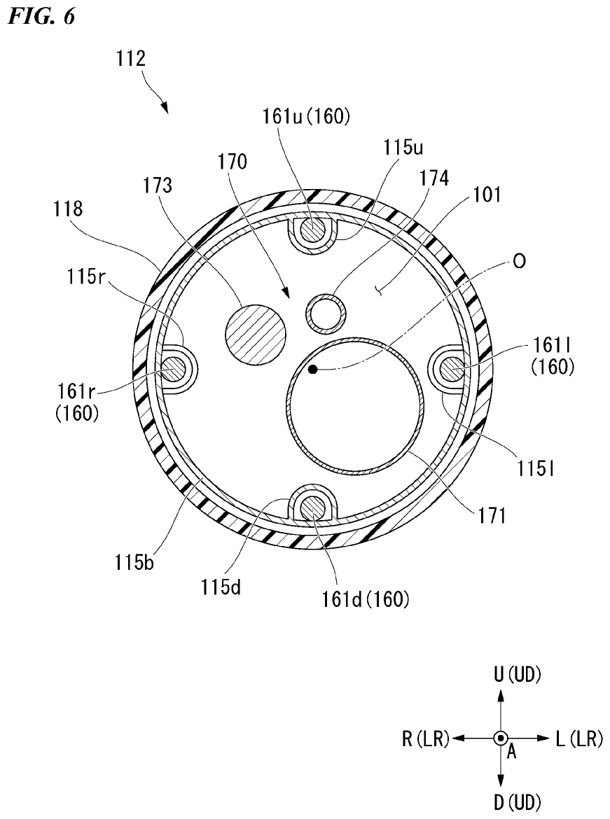

FIG. 6 is a cross-sectional view of the curved portion 112 along the line C1-C1 of FIGS. 4 and 5.

An upper wire guide 115u and a lower wire guide 115d are formed on the inner peripheral surface of the second node ring 115b. The upper wire guide 115u and the lower wire guide 115d are arranged on both sides in the UD direction with the central axis O in the longitudinal direction A interposed therebetween. A left wire guide 1151 and a right wire guide 115r are formed on the inner peripheral surface of the first node ring 115a. The left wire guide 1151 and the right wire guide 115r are arranged on both sides in the LR direction with the central axis O in the longitudinal direction A interposed therebetween.

A through-hole through which the curved wire 160 is inserted is formed in the upper wire guide 115u, the lower wire guide 115d, the left wire guide 1151, and the right wire guide 115r along the longitudinal direction A.

The curved wire 160 is a wire that bends the curved portion 112. The curved wire 160 extends through the internal path 101 to the connector 240. As shown in FIGS. 4 and 6, the curved wire 160 has an upper curved wire 161u, a lower curved wire 161d, a left curved wire 1611, a right curved wire 161r, and four wire sheaths 161s.

As shown in FIG. 4, separate wire sheaths 161s are inserted into the upper curved wire 161u, the lower curved wire 161d, the left curved wire 1611, and the right curved wire 161r. The distal end of the wire sheath 161s is attached to the nodal ring 115 at the proximal end of the curved portion 112. The wire sheath 161s extends to the connector 240.

The upper curved wire 161u and the lower curved wire 161d are wires that bend the curved portion 112 in the UD direction. The upper curved wire 161u is inserted with the upper wire guide 115u. The lower curved wire 161d is inserted with the lower wire guide 115d.

As shown in FIG. 4, the distal ends of the upper curved wire 161u and the lower curved wire 161d are fixed to the distal end 116 of the distal end of the curved portion 112. The distal ends of the upper curved wire 161u and the lower curved wire 161d fixed to the distal end portion 116 are arranged on both sides in the UD direction with the central axis O in the longitudinal direction A interposed therebetween.

The left curved wire 1611 and the right curved wire 161r are wires that bend the curved portion 112 in the LR direction. The left curved wire 1611 is inserted with the left wire guide 1151. The right curved wire 161r is inserted with the right wire guide 115r.

As shown in FIG. 4, the distal ends of the left curved wire 1611 and the right curved wire 161r are fixed to the distal end 116 of the curved portion 112. The distal ends of the left curved wire 1611 and the right curved wire 161r fixed to the distal end portion 116 are arranged on both sides in the LR direction with the central axis O in the longitudinal direction A interposed therebetween.

The curved portion 112 can be bent in a desired direction by pulling or relaxing the curved wire 160 (upper curved wire 161u, lower curved wire 161d, left curved wire 1611, right curved wire 161r).

As shown in FIG. 6, the curved wire 160, the channel tube 171 and the imaging cable 173, and the light guide 174 are inserted into the internal path 101 formed inside the curved portion 112.

The internal soft part 119 is a long and flexible tubular member. The curved wire 160, the channel tube 171 and the imaging cable 173 and the light guide 174 are inserted into the internal path 101 formed in the internal soft portion 119. At the distal end of the soft portion 119 in the body, a distal end portion 119a formed in a substantially cylindrical shape by a metal or the like is provided.

### [Stand 200]

The stand 200 is a self-standing stand that can be placed at a position away from the trolley 800. The stand 200 includes a pedestal 210, a pole 220, a support plate 230, and the connector 240.

The pedestal 210 is a leg for supporting the pole 220. The pedestal 210 illustrated in FIG. 1 is formed in a disk shape, but the pedestal 210 may have another shape as long as it can support the pole 220. Since the pedestal 210 is formed as a flat plate, for example, the legs of the surgeon S are less likely to get caught and are less likely to get in the way. The pedestal 210 may have a plurality of support legs such as a tripod.

The pole 220 is a support column that stands up from the pedestal 210 and supports the support plate 230. The pole 220 has a length capable of supporting the support plate 230 and the connector 240 at a position higher than the operating table T. The pole 220 may have a height-adjusting mechanism that can adjust the heights of the support plate 230 and the connector 240 according to the height of the operating table T.

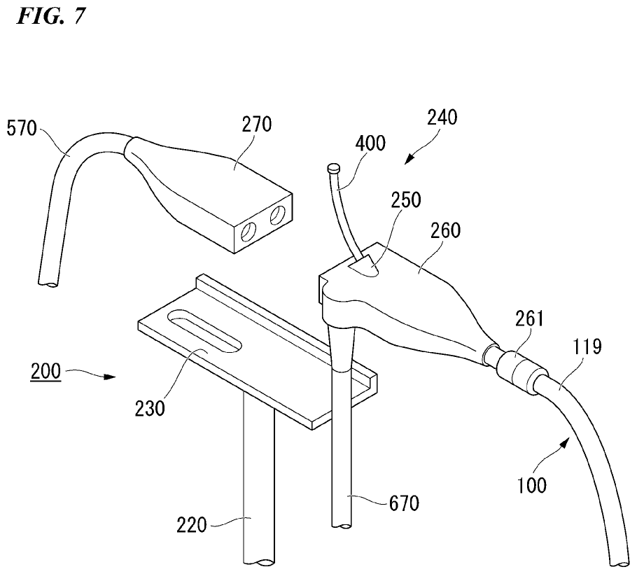

FIG. 7 is a perspective view of the connector 240 before mounting.

The support plate 230 is a flat plate provided on the upper part of the pole 220 and supports the connector 240. The support plate 230 is rotatably attached to the pole 220 in the horizontal direction. The connector 240 supported by the support plate 230 is rotatable in the horizontal direction.

The connector 240 detachably connects the drive cable 570 and the endoscope 100. Further, the image control cable 670 and the endoscope 100 are detachably connected to each other. The connector 240 has a forceps opening 250, a first connector 260, and a second connector 270.

The forceps opening 250 is an insertion port for inserting the treatment tool 400 into the channel tube 171 of the endoscope 100. In this embodiment, the forceps opening 250 is provided on the first connector 260. The treatment tool 400 may be inserted from the forceps opening 250 via the extension channel tube 130 as shown in FIG. 1, or may be inserted directly from the forceps opening 250.

The first connector 260 is a connector to which the proximal end of the internal soft portion 119 of the endoscope 100 is attached. The first connector 260 is integrally connected to the proximal end of the endoscope 100. Specifically, the first connector 260 has a rotary connection connector 261. The rotary connection connector 261 connects the internal soft portion 119 to the first connector 260 so as to be rotatable with respect to the longitudinal axis of the internal soft portion 119. The first connector 260 is detachable from the second connector 270. Further, the first connector 260 is detachable from the distal end of the video control cable 670.

The second connector 270 is a connector to which the distal end of the drive cable 570 is attached. The second connector 270 is connected to the distal end of the drive cable at one end. The second connector 270 is detachable from the first connector 260.

The mounted first connector 260 and second connector 270 can be installed on the support plate 230 as shown in FIG. 1. The first connector 260 and the second connector 270 are fixed so as not to be detachable by installing them on the support plate 230. The first connector 260 and the second connector 270 can be attached and detached by removing them from the support plate 230.

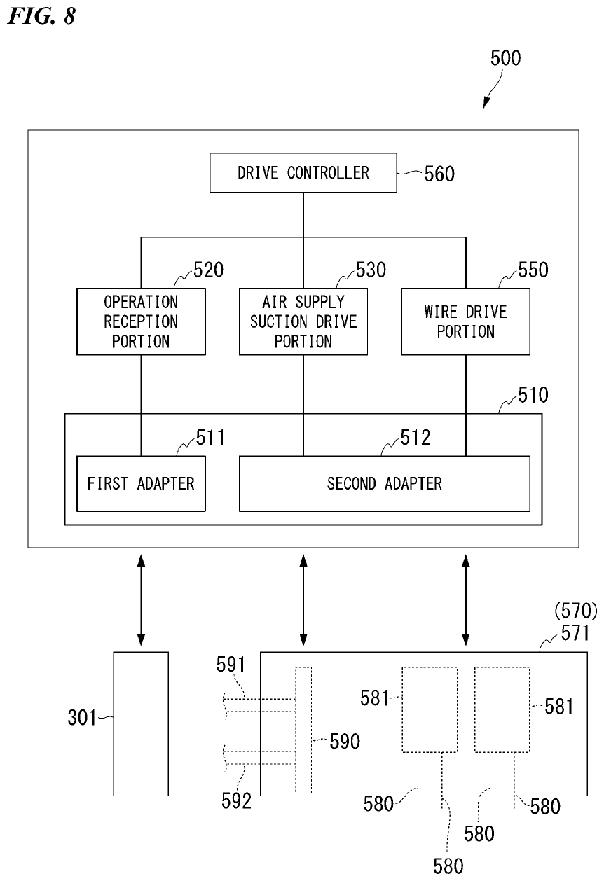

When the first connector 260 and the second connector 270 are attached, the drive wire 580 (see FIG. 8) of the drive cable 570 is connected so that the curved wire 160 of the endoscope 100 can be pulled and relaxed. Further, the air supply suction tube 590 (see FIG. 8) of the drive cable 570 is connected to the channel tube 171 of the endoscope 100.

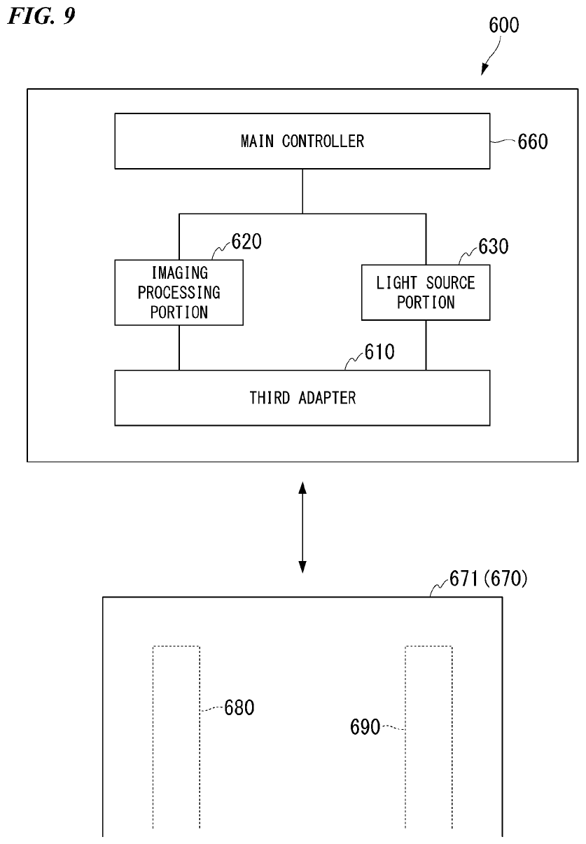

When the video control cable 670 is attached to the first connector 260, the video cable 680 (see FIG. 9) of the video control cable 670 is connected to the imaging cable 173 of the endoscope 100. Further, the light cable 690 (see FIG. 9) of the image control cable 670 is connected to the light guide 174 of the endoscope 100.

The stand 200 can be arranged at a position away from the trolley 800. By arranging the stand 200 at the positions shown in FIGS. 1 and 2, the portion of the flexible portion 119 in the body that is not inserted into the body of the patient P can be bent into a U shape and placed on the operating table T. Further, the proximal end of the internal soft portion 119 is attached to the first connector 260 of the stand 200 at a position higher than the operating table T. Therefore, the load on the patient P due to the weight of the soft portion 119 (insertion portion 110) in the body is small. The longer the insertion portion 110 is, the more remarkable this effect is.

The proximal end of the internal soft portion 119 is connected to the first connector 260 by the rotary connector 261. Therefore, the surgeon S can easily rotate (roll) the internal soft portion 119 with respect to the longitudinal axis of the internal soft portion 119. Further, even when the soft portion 119 in the body is rolled, the rotational force due to the roll rotation is not transmitted to the proximal end side of the first connector 260. Therefore, even when the operator S rolls the soft portion 119 in the body, the stand 200 can maintain an independent state without falling.

### [Operation Device 300]

The operation device 300 is a device for inputting an operation for driving the endoscope 100. The input operation input is transmitted to the drive device 500 via the operation cable 301.

### [Drive 500]

FIG. 8 is a functional block diagram of the drive device 500.

The drive device 500 includes an adapter 510, an operation receiving portion 520, an air supply suction drive portion 530, a wire drive portion 550, and a drive controller 560.

The adapter 510 has a first adapter 511 and a second adapter 512. The first adapter 511 is an adapter to which the operation cable 301 is detachably connected. The second adapter 512 is an adapter to which the connecting portion 571 provided on the proximal end side of the drive cable 570 is detachably connected. The drive cable 570 includes a drive wire 580 and an air supply suction tube 590.

The operation receiving portion 520 receives an operation input from the operation device 300 via the operation cable 301. When the operation device 300 and the drive device 500 communicate by wireless communication instead of wired communication, the operation receiving portion 520 has a known wireless receiving module.

The air supply suction drive portion 530 is connected to the air supply suction tube 590 of the drive cable 570. The air supply suction drive portion 530 includes a pump or the like, and supplies air or liquid to the air supply suction tube 590. Further, the air supply suction drive portion 530 sucks air from the air supply suction tube 590.

A water supply tube 591 and a drain tube 592 are attached to the air supply suction tube 590. The water supply tube 591 is connected to the water supply tank 820, and supplies a liquid such as sterile water used for water supply from the water supply tank 820 to the air supply suction tube 590. The drain tube 592 is connected to the drain tank 830, and the blood or the like discharged to the outside of the body by suction by the endoscope 100 is discharged from the air supply suction tube 590 to the drain tank 830.

The wire drive portion 550 is connected to the drive wire 580 of the drive cable 570 via a drive wire adapter 581. The wire drive portion 550 has a drive portion and an encoder (not shown). The drive portion pulls or relaxes the drive wire 580 of the drive cable 570 by a pulley or the like. The encoder detects the traction amount of the drive wire 580. The detection result of the encoder is acquired by the drive controller 560 of the drive device 500.

The drive controller 560 controls the entire drive device 500. The drive controller 560 acquires the operation input received by the operation receiving portion 520. The drive controller 560 controls the air supply suction drive portion 530 and the wire drive portion 550 based on the acquired operation input.

The drive controller 560 is a computer capable of executing a program including a processor, a memory, a storage portion capable of storing programs and data, and an input/output controller. The function of the drive controller 560 is realized by the processor executing the program. At least some functions of the drive controller 560 may be realized by a dedicated logic circuit.

The drive controller 560 may further have a configuration other than the processor, the memory, the storage portion, and the input/output controller. For example, the drive controller 560 may further have an image calculation portion that performs a part or all of image processing and image recognition processing. By further having an image calculation portion, the drive controller 560 can execute specific image processing and image recognition processing at high speed. The image calculation portion may be mounted on a separate hardware device connected by a communication line.

### [Video Control Device 600]

FIG. 9 is a functional block diagram of the video control device 600.

The video control device 600 includes a third adapter 610, an imaging-processing portion 620, a light source portion 630, and a main controller 660.

The third adapter 610 is an adapter to a the connecting portion 671 provided on the proximal end side of the video control cable 670 is detachably connected. The video control cable 670 includes a video cable 680 and a light cable 690.

The imaging-processing portion 620 is connected to the video cable 680. The imaging-processing portion 620 converts the imaging signal acquired from the imaging portion 111c of the distal end portion 111 into an imaging image via the imaging cable 173 and the video cable 680.

The light source portion 630 is connected to the light cable 690. The light source portion 630 generates illumination light to be applied to the imaging target. The illumination light generated by the light source portion 630 is guided to the illumination portion 111b of the distal end portion 111 via the light cable 690 and the light guide 174.

The main controller 660 is a computer capable of executing a program equipped with a processor, memory, and the like. The function of the main controller 660 is realized by the processor executing the program. At least some functions of the main controller 660 may be realized by a dedicated logic circuit.

The main controller 660 can perform image processing on the captured image acquired by the imaging-processing portion 620. The main controller 660 can generate a GUI image or a CG image for the purpose of providing information to the surgeon S. The main controller 660 can display the captured image, GUI image, and CG image on the display device 900.

The main controller 660 is not limited to an integrated hardware device. For example, the main controller 660 may be configured by partially separating it as a separate hardware device and then connecting the separated hardware device via a communication line. For example, the main controller 660 may be a cloud system that connects the separated storage portions 563 with a communication line.

The main controller 660 may further have a configuration other than the processor and the memory. For example, the main controller 660 may further have an image calculation portion that performs a part or all of the image processing and the image recognition processing performed by the processor. By further having an image calculation portion, the main controller 660 can execute specific image processing and image recognition processing at high speed. The image calculation portion may be mounted on a separate hardware device connected by a communication line.

### [Operation of Endoscope System 1000]

Next, the operation and operation of the endoscope system 1000 of the present embodiment will be described. Specifically, a procedure for observing and treating an affected area formed on a tube wall in the large intestine using the endoscope system 1000 will be described.

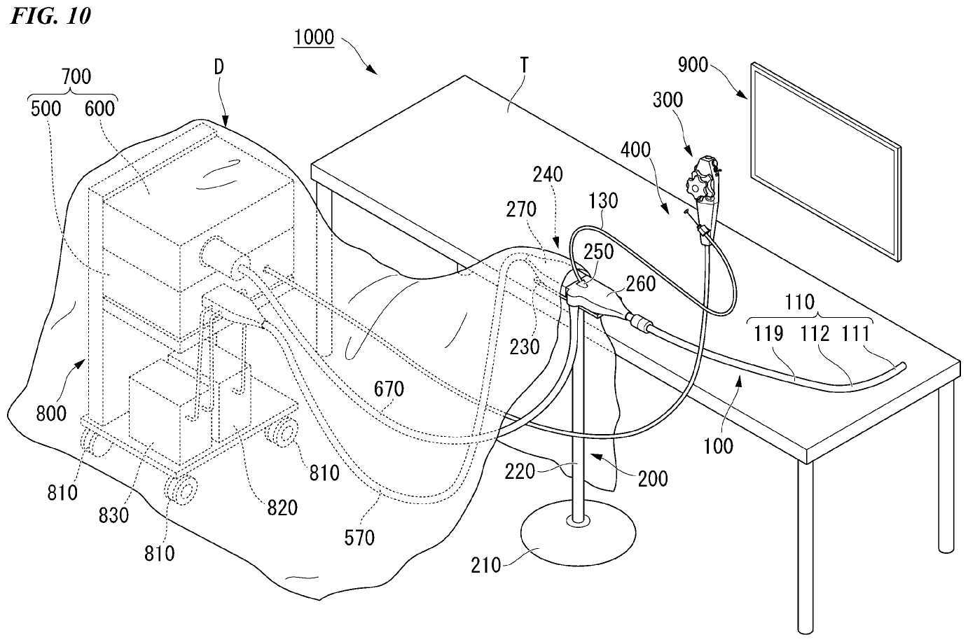

FIG. 10 is a diagram showing the trolley 800 or the like to which a drape D is applied.

Before starting the observation and treatment, the surgeon S or an assistant applies the drape D to an unclean area that does not require treatment such as disinfection after the observation and treatment. Specifically, the drape D is applied to the trolley 800, the drive cable 570, the video control cable 670, and the second connector 270, which are unclean areas.

The surgeon S inserts the insertion portion 110 of the endoscope 100 into the large intestine from the anus of the patient P from the distal end. As shown in FIG. 2, the surgeon S moves the insertion portion 110 to bring the distal end portion 111 closer to the affected area while observing the captured image displayed on the display device 900, and while operating the internal soft portion 119 with the right hand R. Further, the surgeon S operates the operation device 300 with the left hand L to bend the curved portion 112 as necessary.

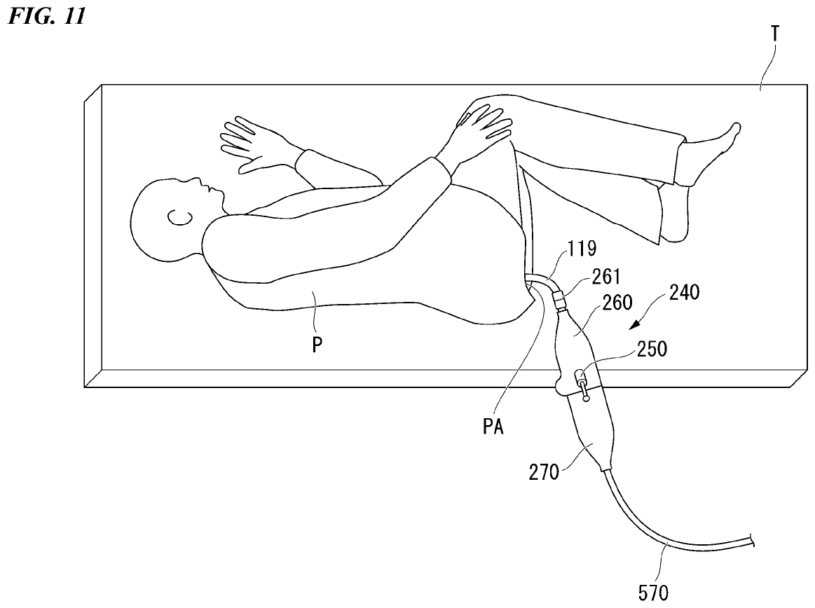

FIG. 11 is a diagram showing the connector 240 arranged near the patient P.

The stand 200 and the connector 240 can be located away from the trolley 800 and close to the patient P and the operator S. Therefore, the surgeon S can easily change the position and posture of the internal soft portion 119 by rotating the connector 240 in the horizontal direction by operating the internal soft portion 119 of the endoscope 100 with the right hand R. Since the connector 240 is supported higher than the operating table T, the first connector 260 can be brought closer to the patient P's anus, as shown in FIG. 11. By bringing the first connector 260 close to the anal PA of the patient P, the insertion portion 110 of the endoscope 100 can be effectively utilized up to the proximal end side, so that the total length of the insertion portion 110 of the endoscope 100 can be shortened.

The surgeon S or the assistant reprocesses (cleans) a clean area that requires treatment such as disinfection after observation and treatment. Specifically, the endoscope 100 and the first connector 260, which are clean areas, are reprocessed (cleaned). The endoscope system 1000 is arranged at a position where the endoscope 100 and the drive device 500 for electrically driving the endoscope 100 are separated from each other, but can be divided at the connector 240, which is an intermediate point between the endoscope 100 and the drive device 500. Therefore, only the endoscope 100 and the first connector 260, which are clean areas, can be reprocessed (cleaned). As a result, the number of members to be reprocessed (cleaned) can be reduced.

According to the endoscope system 1000 according to the present embodiment, handling and reprocessing (cleaning) such as observation and treatment using the endoscope 100 can be easily performed.

Although the first embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

### Second Embodiment

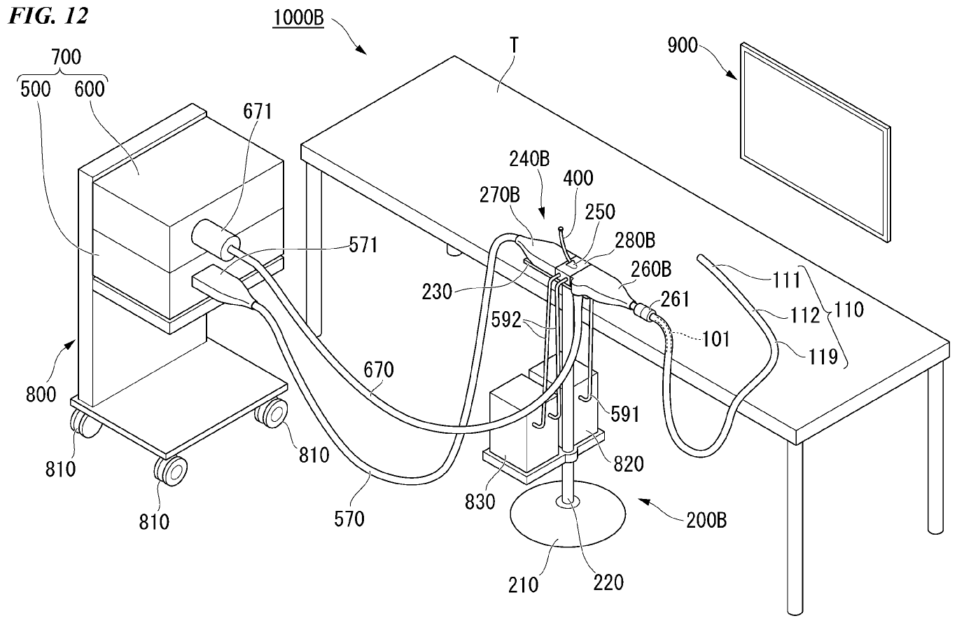

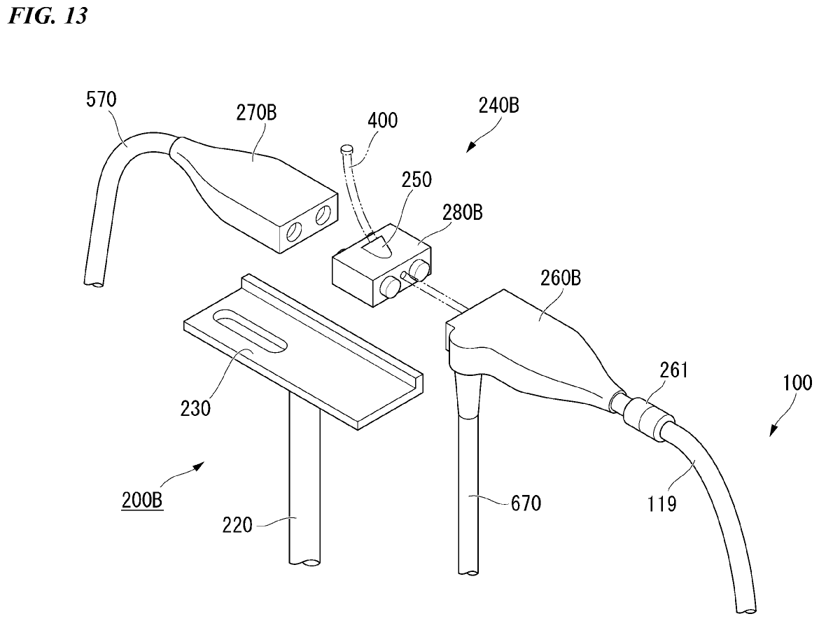

The second embodiment of the present invention will be described with reference to FIGS. 12 and 13. In this embodiment, the aspect of the stand 200 is different from that of the first embodiment. In the following description, the reference numerals will be given to the configurations common to those already described, and duplicate description will be omitted.

FIG. 12 is an overall view of an endoscope system 1000B according to the second embodiment. The endoscope system 1000B includes the endoscope 100, a stand 200B, the operation device 300, the treatment tool 400, the drive device 500, the video control device 600, the trolley 800, and the display device 900. In the present embodiment, the water supply tank 820 and the drain tank 830 are arranged near the stand 200B. In FIG. 12, the operation device 300 is not shown.

### [Stand 200B]

The stand 200B is a self-standing stand that can be placed at a position away from the trolley 800. The stand 200B includes a pedestal 210, the pole 220, the support plate 230, and a connector 240B.

FIG. 13 is a perspective view of the connector 240B before mounting.

The connector 240B detachably connects the drive cable 570 and the endoscope 100. Further, the image control cable 670 and the endoscope 100 are detachably connected to each other. The connector 240B has a forceps opening 250, a first connector 260B, a second connector 270B, and a third connector 280B. In this embodiment, the forceps opening 250 is provided on the third connector 280B.

The first connector 260B is a connector to which the proximal end of the internal soft portion 119 of the endoscope 100 is attached. The first connector 260B is detachable from the third connector 280B. Further, the first connector 260B is detachable from the distal end of the video control cable 670.

The second connector 270B is a connector to which the distal end of the drive cable 570 is attached. The second connector 270B is detachable from the third connector 280B.

The third connector 280B detachably connects the first connector 260B and the second connector 270B. That is, the first connector and the second connector are detachably connected via the third connector. A water supply tube 591 and a drain tube 592 are attached to the third connector 280B. The water supply tube 591 is connected to the water supply tank 820, and supplies a liquid such as sterile water used for water supply from the water supply tank 820 to the channel tube 171 or the air supply suction tube 590. The drain tube 592 is connected to the drain tank 830, and the blood or the like discharged to the outside of the body by suction by the endoscope 100 is discharged from the channel tube 171 or the air supply suction tube 590 to the drain tank 830. The water supply is not limited to sending water into the body, and may include sending a chemical solution or the like into the body.

The first connector 260 and the second connector 270 connected to the third connector 280B can be installed on the support plate 230 as shown in FIG. 12. The first connector 260B, the second connector 270B, and the third connector 280B are fixed so as not to be detachable by installing them on the support plate 230. The first connector 260B and the second connector 270B can be attached to and detached from the third connector 280B by removing them from the support plate 230.

When the first connector 260 and the second connector 270 are attached via the third connector 280B, the drive wire 580 of the drive cable 570 is connected so that the curved wire 160 of the endoscope 100 can be pulled and relaxed. Further, the air supply suction tube 590 of the drive cable 570 is connected to the channel tube 171 of the endoscope 100.

When the video control cable 670 is attached to the first connector 260 via the third connector 280B, the video cable 680 of the video control cable 670 is connected to the imaging cable 173 of the endoscope 100. Further, the light cable 690 of the image control cable 670 is connected to the light guide 174 of the endoscope 100.

According to the endoscope system 1000B according to the present embodiment, observation and treatment and reprocessing (cleaning) using the endoscope 100 can be easily performed. By arranging the third connector 280B between the first connector 260B located in the clean area and the second connector 270B located in the unclean area, the unclean area and the clean area can be separated more safely. Further, the water supply tank 820 and the drain tank 830 can be arranged in a clean area, and the unclean area can be maintained in a state where cleaning is not required.

Although the second embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

### Third Embodiment

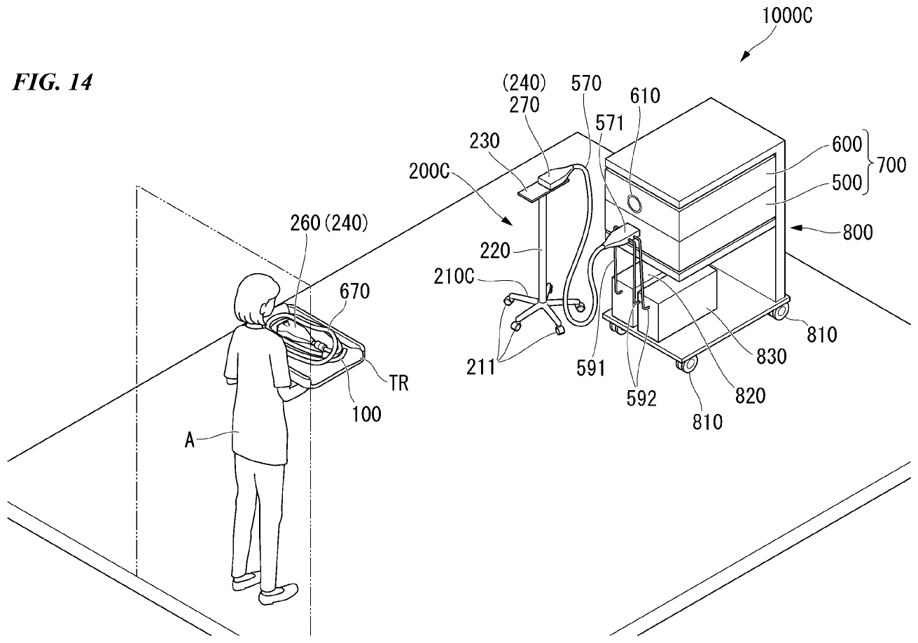

The third embodiment of the present invention will be described with reference to FIGS. 14 to 24. In this embodiment, the aspect of the stand 200 is different from that of the first embodiment. In the following description, the reference numerals will be given to the configurations common to those already described, and duplicate description will be omitted.

FIGS. 14 to 22 are diagrams showing a way of using an endoscope system 1000C according to the present embodiment. The endoscope system 1000C includes the endoscope 100, a stand 200C, the operation device 300, the treatment tool 400, the drive device 500, the video control device 600, the trolley 800, and the display device 900.

The stand 200C is a self-standing stand that can be placed at a position away from the trolley 800. The stand 200C includes a support leg 210C having wheels (casters) 211, the pole 220, the support plate 230, and the connector 240. Since the stand 200C has wheels 211, the user can easily move the stand 200C when preparing the device before the operation or when changing the layout of the device during the operation. The support leg 210C may be provided with a stopper capable of stopping the rotational operation of the wheel 211.

### [Operation of Endoscope System 1000C]

Next, the operation and operation of the endoscope system 1000C of the present embodiment will be described. Specifically, preparation of the endoscope system 1000C, a procedure for observing and treating the affected area formed on the tube wall in the large intestine using the endoscope system 1000C, and tidying and cleaning of the endoscope system 1000C will be explained.

### <Step S1: Equipment Delivery>

As shown in FIG. 14, the assistant A brings the endoscope 100, the first connector 260, and the image control cable 670 housed in the tray TR into the operating room. The endoscope 100, the drive cable 570, and the image control cable 670 are detachable by the connector 240. The assistant A can individually carry the separated endoscope 100 and the like into the operating room. Therefore, the load due to the weight of the devices carried in at one time is reduced.

### <Step S2: System Startup>

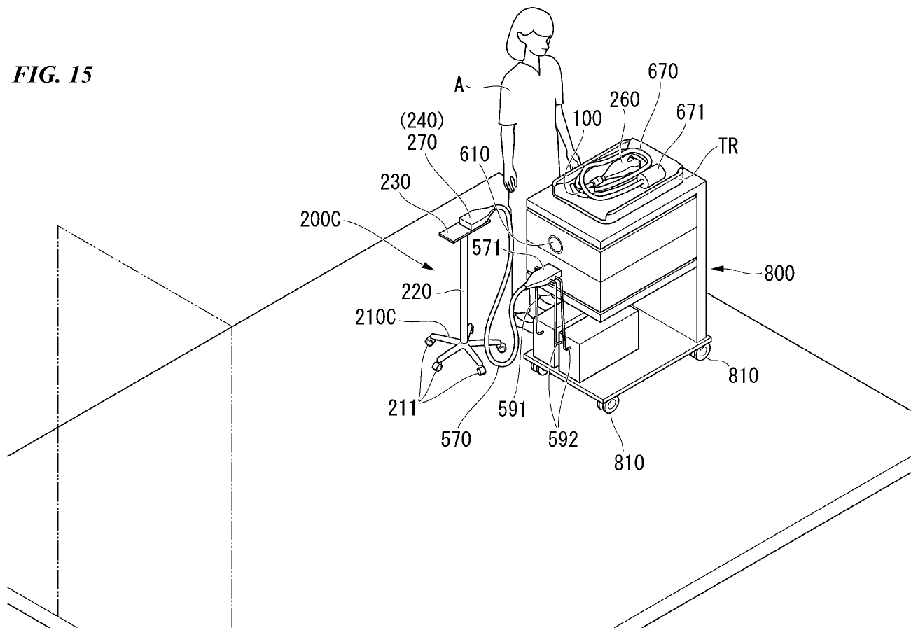

As shown in FIG. 15, the assistant A activates the endoscope system 1000C after placing the tray TR on the trolley 800. In addition, the assistant A registers preoperative information such as patient information in the endoscope system 1000C.

### <Step S3: Endoscope Connection>

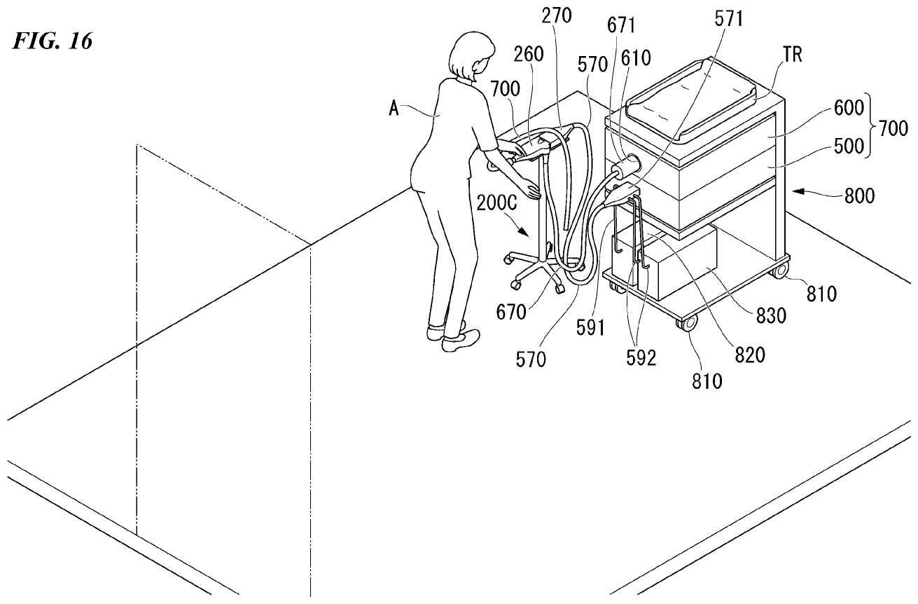

As shown in FIG. 16, the assistant A connects the endoscope 100 to the drive cable 570 by connecting the first connector 260 to the second connector 270. Further, the assistant A connects the video control cable 670 to the video control device 600 by connecting the connecting portion 671 of the video control cable 670 to the third adapter 610. The assistant A can install the endoscope 100 in a folded state by hooking it on the stand 200C.

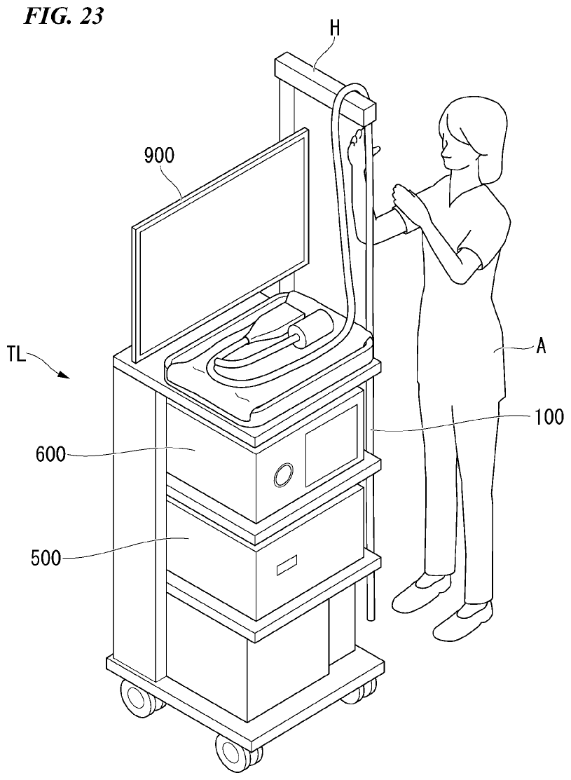

FIG. 23 is a diagram showing a trolley TL used in a conventional endoscope system. In the conventional endoscope system, the assistant A needs to hang the endoscope 100 on the hanger H provided at a high position of the trolley TL. On the other hand, in the endoscope system 1000C, the assistant A only needs to hook the endoscope 100 on the stand 200C and does not need to hang the endoscope 100 on the hanger H provided at a high position.

### <Step S4: Other Connections>

The assistant A makes other connections necessary for using the endoscope system 1000C. For example, the assistant A connects the display cable to the display device 900 and the drain tube 592 to the drain tank 830.

### <Step S5: Patient Delivery>

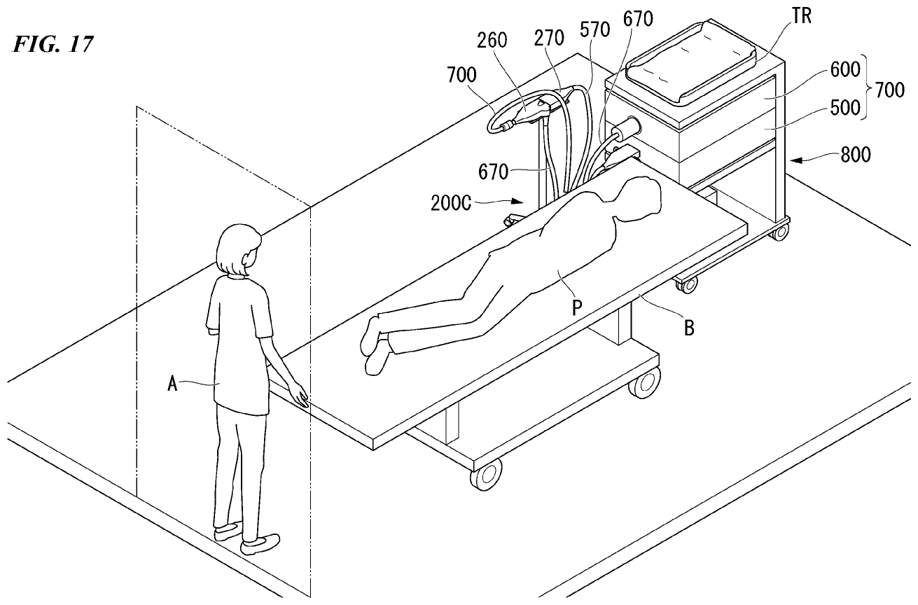

As shown in FIG. 17, the assistant A brings the bed B on which the patient P is placed into the operating room.

### <Step S6: Insertion of Endoscope>

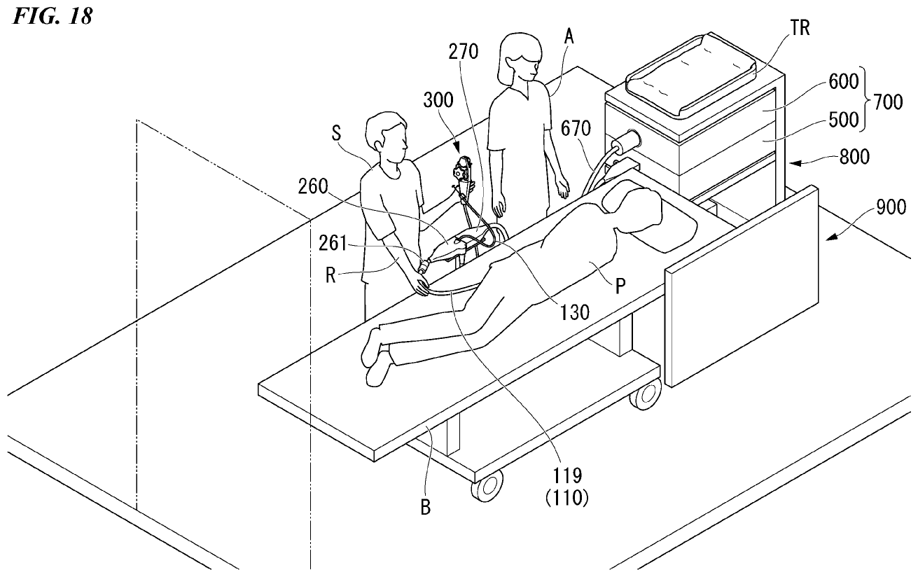

As shown in FIG. 18, the surgeon S inserts the insertion portion 110 of the endoscope 100 from the distal end into the large intestine from the anus of the patient P. While observing the captured image displayed on the display device 900, the surgeon S moves the insertion portion 110 while operating the internal soft portion 119 with the right hand R to bring the distal end portion 111 closer to the affected area.

The stand 200C and trolley 800 are small and easy to move. Therefore, it is easy to arrange the stand 200C and the trolley 800 at appropriate positions, and it is easy to secure a sufficient work area for the operator S and the assistant A. For example, as shown in the first embodiment, the stand 200C can be arranged near the bed B and on the left side of the operator S so that the endoscope 100 can be easily handled (see FIG. 2). The trolley 800 can be placed on the head side of the patient P so as not to interfere with the procedure. On the other hand, the trolley TL used in the conventional endoscopic system shown in FIG. 23 is large and difficult to move during surgery.

### <Step S7: Observation/Biopsy>

The surgeon S operates the operation device 300 and the like to observe the affected area and perform a biopsy with the endoscope 100.

### <Step S8: Biopsy Tissue Storage>

When a biopsy is performed, the surgeon S takes out the biopsied tissue from the body and stores it.

### <Step S9: Endoscope Removal>

The surgeon S removes the insertion portion 110 of the endoscope 100 from the body of the patient P.

### <Step S10: Patient Leaving>

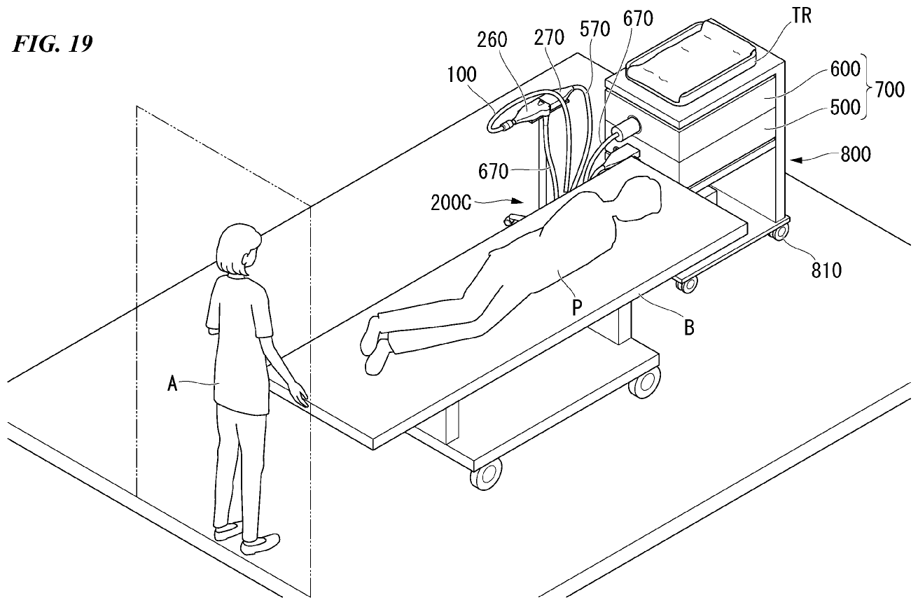

As shown in FIG. 19, the assistant A hooks the removed endoscope 100 on the stand 200C and puts it in a folded state. The assistant A moves the stand 200C closer to the trolley 800. After that, the assistant A leaves the patient P on the bed B from the operating room. The assistant A can hook the endoscope 100 on the stand 200C and place it, and can easily move the stand 200C on which the endoscope 100 is hooked. Therefore, the work load required to remove the patient P from the operating room is reduced.

### <Step S11: Bedside Cleaning>

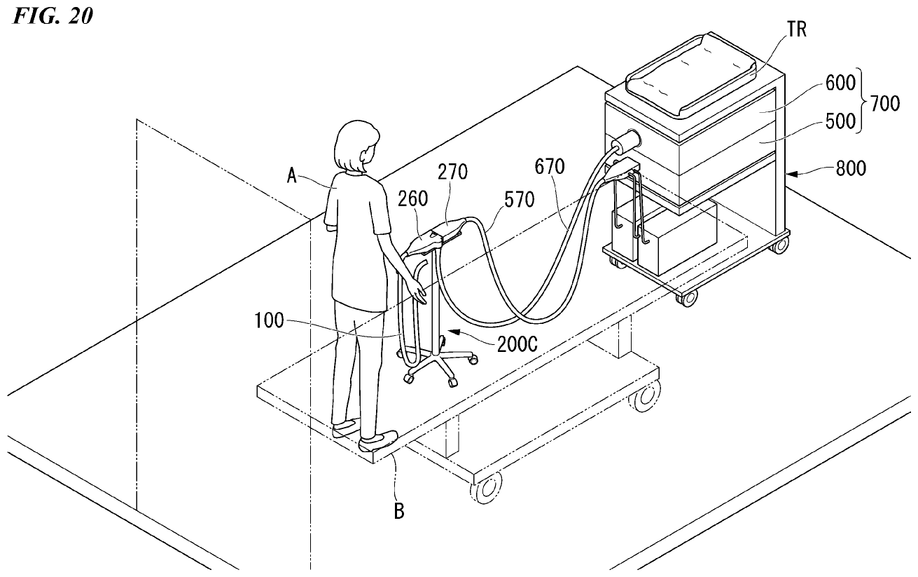

As shown in FIG. 20, the assistant A cleans the vicinity of the position where the bed B was. The stand 200C and trolley 800 are small and easy to move. Therefore, the assistant A can easily wash the part requiring cleaning by moving the stand 200C and the trolley 800. On the other hand, the trolley TL used in the conventional endoscope system shown in FIG. 23 is large and difficult to move.

### <Step S12: Disconnection>

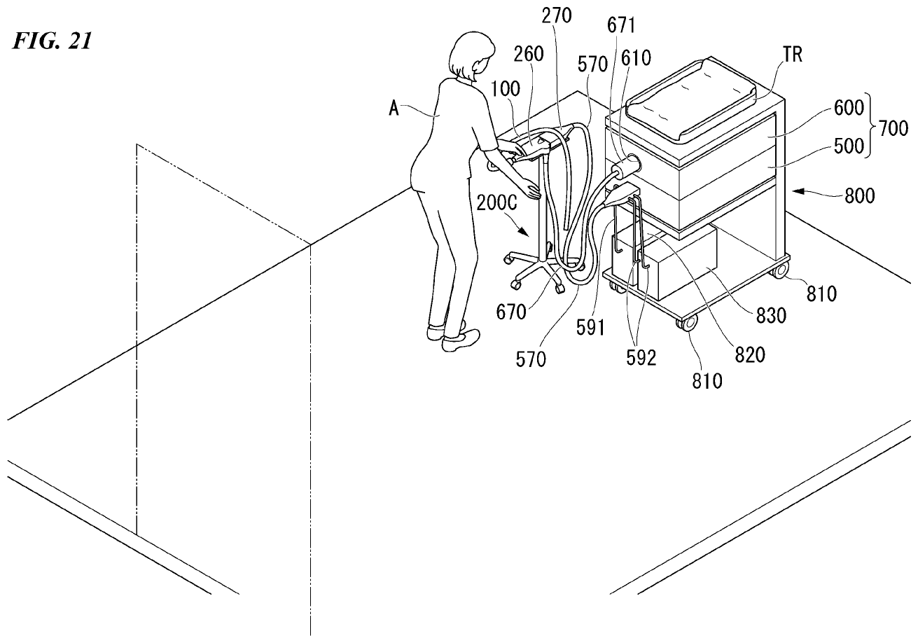

As shown in FIG. 21, the assistant A disconnects the cables and tubes between the devices. For example, the assistant A removes the first connector 260 from the second connector 270. Further, the assistant A removes the drain tube 592 from the drain tank 830. Next, the assistant A cleans the drain tank 830.

### <Step S13: Disconnection>

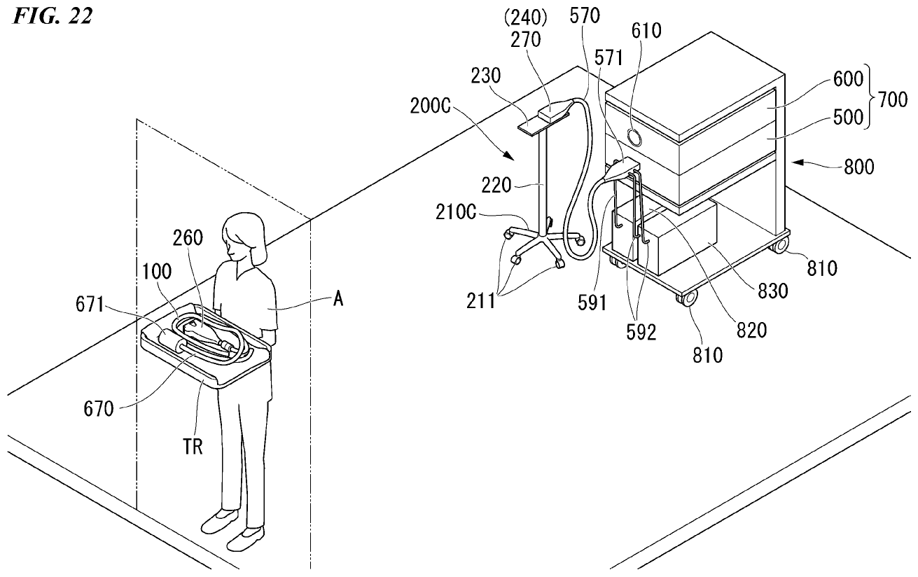

As shown in FIG. 22, the assistant A accommodates the endoscope 100, the first connector 260, and the image control cable 670 in the tray TR and carries them out of the operating room. The assistant A can individually carry out the separated endoscope 100 and the like from the operating room. Therefore, the load due to the weight of the device to be carried out at one time is reduced.

### <Step S14: Reprocess>

The assistant A reprocesses (cleans) the clean area that requires treatment such as disinfection. Specifically, the endoscope 100, which is a clean area, and the first connector 260 are reprocessed. The endoscope system 1000 is arranged at a position where the endoscope 100 and the drive device 500 for electrically driving the endoscope 100 are separated from each other, but can be divided at the connector 240, which is an intermediate point between the endoscope 100 and the drive device 500. Therefore, only the endoscope 100 and the first connector 260, which are clean areas, can be reprocessed. As a result, the number of parts to be reprocessed can be reduced.

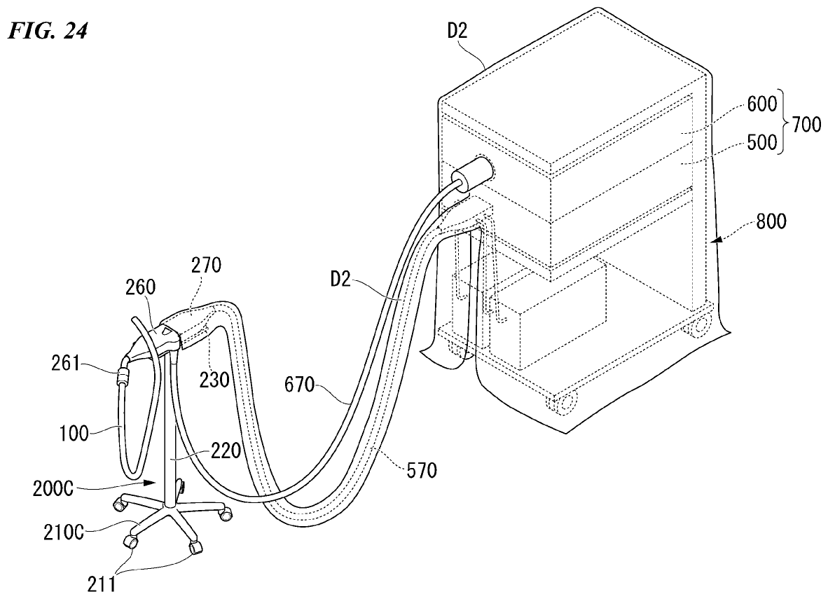

FIG. 24 is a diagram showing the trolley 800 or the like to which a drape D2 is applied.

The drape D2 is hung on the trolley 800, the drive cable 570, and the second connector 270, which are unclean areas. In this case, the drape D2 is not applied to the video control cable 670, and the video control cable 670 is treated as a clean area that requires reprocessing. The drive cable 570 basically does not need to be touched during the operation. Therefore, the postoperative reprocessing process can be simplified by applying the drape D2 for separation. On the other hand, the image control cable 670 may be contaminated via the endoscope 100 and the first connector 260, which are clean areas. Alternatively, the image control cable 670 may be treated as one unit together with the endoscope 100 and the connector 260. Therefore, the image control cable 670 is treated as a clean area where postoperative reprocessing is indispensable, like the endoscope 100.

The degree of reprocessing may be set step by step for each device. For example, the degree of reprocessing may be set so that the degree of reprocessing of the endoscope 100>the degree of reprocessing of the image control cable 670>the degree of reprocessing of the drive cable 570. The degree of reprocessing of the endoscope 100 inserted into the body is the highest. In addition, the degree of reprocessing of the image control cable 670, which has a high possibility of body fluid adhering during the operation via the endoscope 100, is the second highest, and the degree of reprocessing of the drive cable 570, which is unlikely to be touched during surgery, is the third highest. Not limited to the examples given here, it is possible to appropriately change the degree of reprocessing depending on the system configuration, arrangement, and the like. Further, by applying the drape to the region where the degree of reprocessing is relatively low, the postoperative reprocessing process can be simplified.

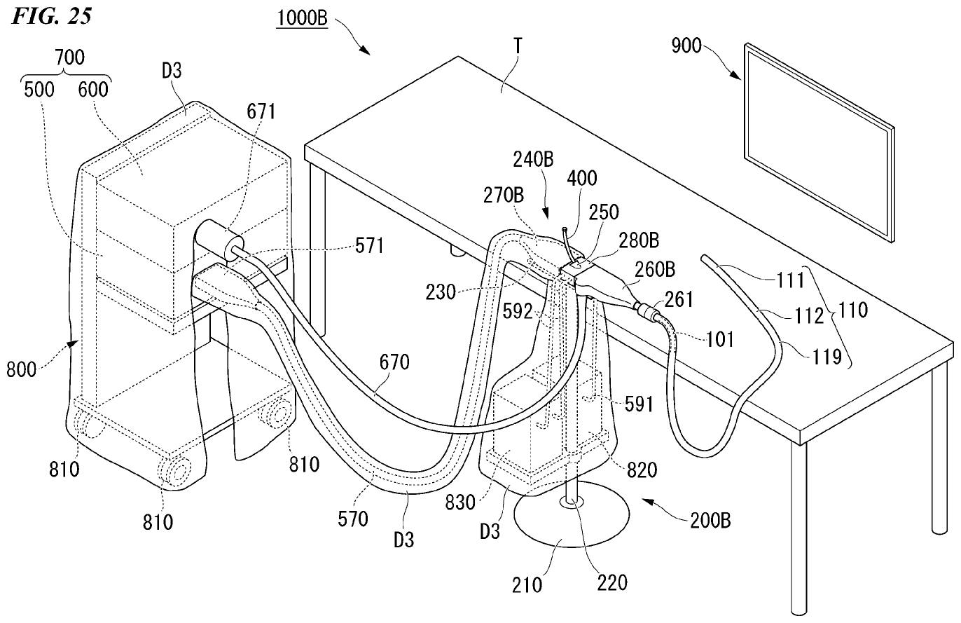

FIG. 25 is a diagram showing the endoscope system 1000B to which a drape D3 is applied.

In the endoscope system 1000B of the second embodiment, the water supply tank 820 and the drain tank 830 are arranged near the stand 200B. In this case, the drape D3 is hung on, for example, the drive cable 570, the support plate 230, the second connector 270B, the water supply tank 820, and the drain tank 830.

According to the endoscope system 1000C according to the present embodiment, preparation of the endoscope system 1000C, handling such as observation and treatment using the endoscope system 1000C, and tidying and cleaning of the endoscope system 1000C can be easily performed.

Although the third embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

Although the procedure in which the treatment target is the large intestine is described in detail in the above embodiment, the treatment target of the procedure using the endoscope system is not limited to this. The procedure using the endoscopic system can target any body cavity including the upper gastrointestinal tract, the lower gastrointestinal tract, and the like.

What is claimed is:

1. An endoscope system comprising:
a drive cable;
an endoscope driven by the drive cable that has been connected;
a drive device that electrically drives the connected drive cable;
a trolley equipped with the drive device; and
a stand that supports a connector that detachably connects the drive cable and the endoscope, and is capable of being installed at a position away from the trolley.

2. The endoscope system according to claim 1, further comprising:
a video cable; and
a video control device that acquires an image captured by the endoscope from the connected image cable,
wherein the trolley is equipped with the drive device and the video control device, and
the connector connects the endoscope and the video cable.

3. The endoscope system according to claim 1, wherein the connector is provided with a forceps opening into which a treatment tool can be inserted into a lumen of the endoscope.

4. The endoscope system according to claim 1, wherein the connector includes a first connector integrally connected to a proximal end of the endoscope and a second connector integrally connected to a distal end of the drive cable, and the first connector and the second connector are detachably connected to each other.

5. The endoscope system according to claim 4, further comprising:
a video cable; and
a video control device that acquires an image captured by the endoscope from the connected image cable,
wherein the trolley is equipped with the drive device and the video control device, and
the first connector connects the endoscope and the video cable.

6. The endoscope system according to claim 5, wherein the first connector is provided with a forceps opening into which a treatment tool can be inserted into a lumen of the endoscope.

7. The endoscope system according to claim 5, wherein the connector further includes a third connector configured to be detachably connected to each of the first connector and the second connector, and the first connector and the second connector are detachably connected via the third connector.

8. The endoscope system according to claim 7, wherein the third connector is provided with a forceps opening into which a treatment tool can be inserted into a lumen of the endoscope.

9. The endoscope system according to claim 8, wherein
the endoscope system further includes a tank that stores liquid for sending water from the endoscope into a body or liquid discharged to outside of the body by suction by the endoscope, and
the third connector connects the tank to a tube for water supply or suction of the endoscope.

10. The endoscope system according to claim 9, wherein the tank is placed on the stand.

11. The endoscope system according to claim 1, wherein
the stand is equipped with a support plate, and
the support plate is detachably connected to the connector and is a flat plate for supporting the connector.

12. The endoscope system according to claim 11, wherein
the stand is further equipped with a pole for supporting the support plate, and
the support plate is rotatably attached to the pole.

13. The endoscope system according to claim 12, wherein the support plate is rotatably attached to the pole in a horizontal direction.

14. The endoscope system according to claim 12, wherein the pole has a length capable of supporting the connector at a position higher than an operating table, or can be extended to a length capable of pointing the connector to a position higher than the operating table.

15. An endoscope comprising:
an insertion portion to be inserted into a body; and
a first connector arranged at a proximal end of the insertion portion, detachably connected to a second connector arranged on a drive cable extending from a drive device for driving the insertion portion, and supported by being detachably connected to the stand.

16. The endoscope according to claim 15, wherein the first connector is detachably connected to a video cable extending from a video control device for acquiring an image captured by the endoscope.

17. The endoscope according to claim 15, wherein the first connector is provided with a forceps opening into which a treatment tool can be inserted into a lumen of the endoscope.

18. The endoscope according to claim 16, wherein the first connector is detachably connected to the second connector with respect to a third connector configured to be detachably connected to each of the first connector and the second connector.

19. The endoscope according to claim 18, wherein the first connector detachably connects a forceps opening into which a treatment tool can be inserted into a lumen of the endoscope and the lumen via the third connector.

20. The endoscope according to claim 18, wherein the first connector connects a tank for storing liquid for sending water from the endoscope into a body or liquid discharged to outside of the body by suction by the endoscope with a tube for water supply or suction of the endoscope via the third connector.
